(19) United States
(12) Patent Application Publication
(10) Pub. No.: US 2023/0144028 A1
(43) Pub. Date: May 11, 2023

(54) RADIOGRAPHIC IMAGING APPARATUS AND METHOD FOR MANUFACTURING THE SAME

(71) Applicant: CANON KABUSHIKI KAISHA, Tokyo (JP)

(72) Inventor: Takashi Fukushima, Kanagawa (JP)

(21) Appl. No.: 18/048,374

(22) Filed: Oct. 20, 2022

(30) Foreign Application Priority Data
Nov. 11, 2021 (JP) 2021-183885

(51) Int. Cl. A61B 6/00

(52) U.S. Cl. CPC A61B 6/4208; A61M 2025/0056

(57) ABSTRACT

A radiographic imaging apparatus includes a radiation detector configured to convert incident radiation into an electric signal related to a radiographic image, a casing containing the radiation detector, and an antibacterial layer formed on at least part of a surface of the casing. An average thickness of the antibacterial layer is thicker than 0.05 μm and thinner than 0.5 μm.

### BACKGROUND

### Field of the Disclosure

The present disclosure relates to a radiographic imaging apparatus that images a subject using radiation, and a method for manufacturing the radiographic imaging apparatus.

### Description of the Related Art

Radiographic imaging apparatuses, which acquire a radiographic image by detecting an intensity distribution of radiation transmitted through a subject to be imaged, are widely and commonly used in medical diagnostic scenes and industrial non-destructive inspection scenes. Practical examples of the radiographic imaging apparatuses that acquire the radiographic image include imaging apparatuses using a flat panel detector (FPD) having a grid-like arrangement of pixels each including a minute photoelectric conversion element to which a semiconductor process technique is applied and a switching element.

The above-described radiographic imaging apparatuses are used in various scenes at medical sites, and are used not only in general imaging rooms but also during ward rounds and emergency care. The radiographic imaging apparatuses may be used in direct contact with patients in various conditions at medical sites, and thus are often cleaned and disinfected with a disinfectant such as alcohol after being used. However, in some situations, the radiographic imaging apparatuses may be unable to be cleaned or disinfected sufficiently. Application of antibacterial treatment to the radiographic imaging apparatuses in consideration of such situations can be an effective method for reducing an infection risk.

Meanwhile, portable radiographic imaging apparatuses are frequently used while being inserted under the subject such as a patient, and frequently stored in and taken from a box mounted on a ward round trolley, a storeroom, or the like. Thus, the surface of a casing (also called an “exterior”) thereof is required to have rub-fastness. Applying the antibacterial treatment to the casing of such a radiographic imaging apparatus can create a risk that an antibacterial agent that has peeled from the casing may attach to the patient or enter a wound of the patient, for example. Thus, the coating film strength of an antibacterial layer in applying the antibacterial agent to the casing is important.

The size of the radiographic imaging apparatus reaches as large as approximately 460 mm×460 mm in the case of a large one, while the thickness thereof is as extremely thin as approximately 15 mm. Further, a large number of components, such as a radiation detector corresponding to the above-described flat panel detector, a support base supporting the radiation detector, and electric boards, are accommodated within the thickness of approximately 15 mm. Thus, a member large in area but extremely thin in thickness is used to form the casing of the radiographic imaging apparatus.

Further, the radiographic imaging apparatus may be used while being laid under the subject such as a patient or be impacted by being accidentally dropped by a user, and thus is also required to be robust enough to withstand such situations. Further, the radiographic imaging apparatus is desired to be as lightweight as possible in consideration of being carried by the user. For example, carbon fiber reinforced plastic (CFRP) is often employed as a material for the casing satisfying these various characteristics.

Generally, possible methods for obtaining sufficient coating film strength in the antibacterial treatment include a method that fuses particles of the antibacterial agent or the antibacterial agent and a base material using heating treatment, and a method that thermally cures the antibacterial agent by adding a curing agent. However, applying heat to the thin large-area casing (e.g., the casing made of CFRP) may cause a deformation or contraction, leading to a visual defect or damage on the casing of the radiographic imaging apparatus. Thus, it is desirable to apply the antibacterial treatment at room temperatures, but it may be difficult to achieve high coating film strength at room temperatures.

### SUMMARY

Aspects of the present disclosure are directed to providing a radiographic imaging apparatus with antibacterial treatment applied to the surface of a casing thereof with sufficient coating film strength, without impairing the external appearance of the radiographic imaging apparatus.

According to an aspect of the present disclosure, a radiographic imaging apparatus includes a radiation detector configured to convert incident radiation into an electric signal related to a radiographic image, a casing containing the radiation detector, and an antibacterial layer formed on at least part of a surface of the casing. An average thickness of the antibacterial layer is thicker than 0.05 μm and thinner than 0.5 μm.

Further, aspects of the present disclosure include a method for manufacturing the above-described radiographic imaging apparatus.

Further features of the present disclosure will become apparent from the following description of exemplary embodiments with reference to the attached drawings.

### BRIEF DESCRIPTION OF THE DRAWINGS

FIGS. 1A and 1B illustrate an example of an external appearance of a radiographic imaging apparatus according to an exemplary embodiment of the present disclosure.

FIG. 2 illustrates an example of an internal configuration of the radiographic imaging apparatus according to the exemplary embodiment of the present disclosure, in A-A′ cross section in FIG. 1B.

FIGS. 3A to 3E illustrate examples of a configuration at and near a front cover, which is indicated by a broken line box B in FIG. 2, in the radiographic imaging apparatus according to the exemplary embodiment of the present disclosure.

FIG. 4 illustrates an example of a configuration at and near a frame, which is indicated by a broken line box C in FIG. 2, in the radiographic imaging apparatus according to the exemplary embodiment of the present disclosure.

### DESCRIPTION OF THE EMBODIMENTS

An exemplary embodiment of the present disclosure will be described below with reference to the attached drawings. Details of configurations that will be described in the exemplary embodiment of the present disclosure are not limited to those in the specification and the drawings. Further, X rays are desirably used as radiation in the exemplary embodiment of the present disclosure, but the radiation is not limited to X rays and includes, for example, α rays, β rays, and γrays in the exemplary embodiment of the present disclosure.

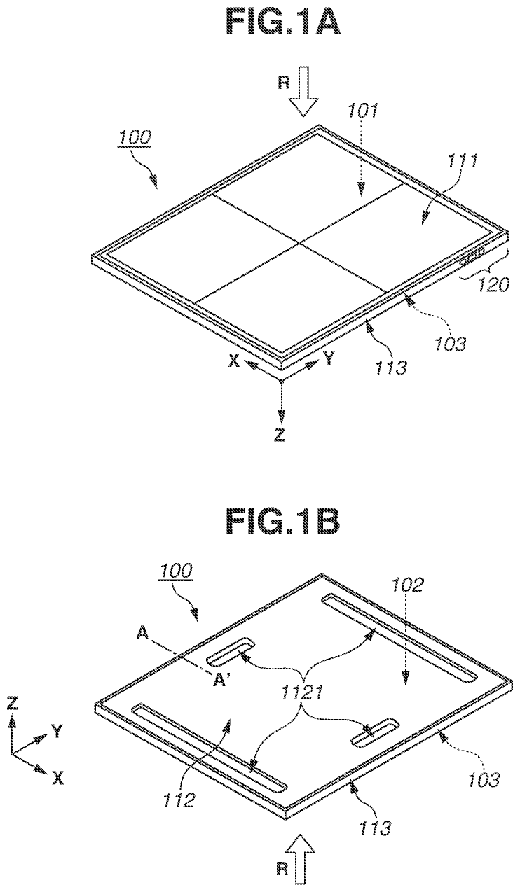

FIGS. 1A and 1B illustrate an example of an external appearance of a radiographic imaging apparatus 100 according to an exemplary embodiment of the present disclosure.

More specifically, FIG. 1A illustrates a radiographic imaging apparatus 100 according to the present exemplary embodiment as viewed from a side where a radiation incident surface 101 on which radiation R is incident is located. FIG. 1A also illustrates an XYZ coordinate system in which a Z direction is a direction in which the radiation R is incident, and an X direction and a Y direction are two directions perpendicular to the Z direction and orthogonal to each other. FIG. 1B illustrates the radiographic imaging apparatus 100 according to the present exemplary embodiment as viewed from a side on which a rear surface 102 is located, which is opposite to the side on which the radiation incident surface 101 illustrated in FIG. 1A is located. FIG. 1B also illustrates an XYZ coordinate system corresponding to the XYZ coordinate system illustrated in FIG. 1A.

As illustrated in FIG. 1A, a front cover 111 is disposed as a member forming the radiation incident surface 101 of the casing 110 of the radiographic imaging apparatus 100. As illustrated in FIG. 1B, a rear cover 112 is disposed as a member forming the rear surface 102 of the casing 110 of the radiographic imaging apparatus 100. Grip portions 1121 are provided in the rear cover 112 so as to enable a user to easily hold the radiographic imaging apparatus 100 with the user's hand, as illustrated in FIG. 1B.

Further, as illustrated in FIG. 1A, a frame 113 is disposed as a member forming a side surface 103, with respect to the radiation incident surface 101, of the casing 110 of the radiographic imaging apparatus 100. The frame 113 is disposed to be interposed between the front cover 111 and the rear cover 112 at the side surface 103 of the casing 110 of the radiographic imaging apparatus 100, and is joined with the front cover 111 and the rear cover 112. Further, as illustrated in FIG. 1A, a user interface 120, including a power switch, a light-emitting diode (LED) indicating a battery remaining amount, a ready switch indicating an imaging preparation state, and a connector for a power cable, is provided on the frame 113.

Further, markings indicating the positions of the center of an imaging region, the user interface 120, and the like are printed on the radiation incident surface 101 side of the front cover 111 of the casing 110 of the radiographic imaging apparatus 100. The markings described here may be provided, for example, by directly painting the markings on the front cover 111 made of carbon fiber reinforced plastic (CFRP) or by sticking an illustrative sheet printed on a sheet material to the front cover 111.

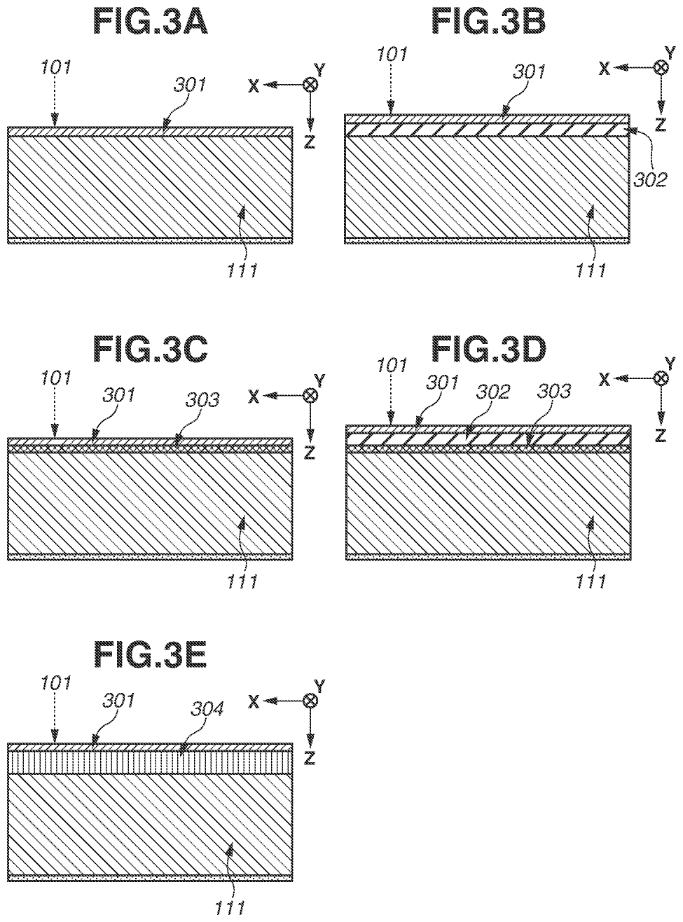

Further, an antibacterial layer 301 (refer to FIG. 3A) is formed on the surface of the casing 110 of the radiographic imaging apparatus 100 by applying an antibacterial agent thereto. In this case, in the present exemplary embodiment, the antibacterial layer 301 may be formed on a part of the surface of the casing 110 of the radiographic imaging apparatus 100 instead of the entire surface of the casing 110 of the radiographic imaging apparatus 100. In other words, the present exemplary embodiment includes a configuration in which the antibacterial layer 301 is formed on at least a part of the surface of the casing 110 of the radiographic imaging apparatus 100. In the present exemplary embodiment, forming the antibacterial layer 301 by applying the antibacterial agent especially to the radiation incident surface 101, which is a contact portion with a patient serving as a subject, the grip portions 1121 to be touched by the user, and/or the like is effective to reduce an infection risk.

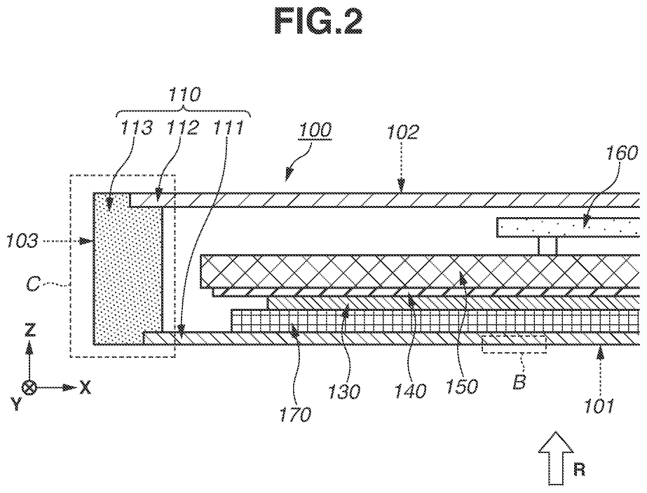

FIG. 2 illustrates an example of an internal configuration of the radiographic imaging apparatus 100 according to the present exemplary embodiment, in A-A′ cross section in FIG. 1B. In FIG. 2, similar components to those illustrated in FIGS. 1A and 1B are denoted by the same reference numerals, and the detailed description thereof will be omitted. FIG. 2 also illustrates an XYZ coordinate system corresponding to the XYZ coordinate system illustrated in FIG. 1B.

As illustrated in FIG. 2, the casing 110 of the radiographic imaging apparatus 100 includes the front cover 111 serving as the member forming the radiation incident surface 101, the rear cover 112 serving as the member forming the rear surface 102, and the frame 113 serving as the member forming the side surface 103. The casing 110 of the radiographic imaging apparatus 100 is formed by these three members (the front cover 111, the rear cover 112, and the frame 113) in the present exemplary embodiment, but may be formed by a member into which these members are integrated.

A radiation detector 130, a radiation shielding sheet 140, a support base 150, a substrate 160, a shock absorption sheet 170, a battery (not illustrated), and the like are contained in the casing 110 of the radiographic imaging apparatus 100 while being arranged at predetermined positions.

The radiation detector 130 is a radiation detection panel that detects the radiation R emitted from a radiation generation apparatus (not illustrated) and transmitted through the subject. More specifically, the radiation detector 130 is a radiation detection panel that detects the radiation R by converting the incident radiation R into an electric signal related to a radiographic image. The electric signal related to the radiographic image that is acquired by the radiation detector 130 is transferred to the outside of the radiographic imaging apparatus 100, and is displayed on a monitor or the like as the radiographic image and used for a diagnosis or the like. The radiation detector 130 is generally formed using a glass substrate, and thus may be broken if receiving a strong impact or load, or a displacement. Thus, the radiation detector 130 is stuck to the support base 150 having high strength and flatness.

The radiation shielding sheet 140 has a function of protecting the substrate 160 such as an electric board from the radiation R transmitted through the subject and the radiation detector 130, and a function of preventing the transmitted radiation R from being incident on the radiation detector 130 again due to reflection or the like.

The support base 150 supports the radiation detector 130 via the radiation shielding sheet 140.

The substrate 160 such as an electric board is arranged closer to the rear surface 102 than the support base 150.

The shock absorption sheet 170 is interposed between the front cover 111 and the radiation detector 130, and used to protect the radiation detector 130 by absorbing an impact received by the casing 110.

Further, the Japanese Industrial Standards (JIS) stipulates that the thickness of the radiographic imaging apparatus 100 is no thicker than approximately 15 mm, depending on the product, and the above-described inner components 130 to 170 are to be contained in the casing 110 having the thin thickness.

If the components arranged between the radiation incident surface 101 and the radiation detector 130 are formed of a substance having a high atomic weight, the transmitted amount of the radiation R reduces, resulting in a failure to acquire a radiographic image with a high-definition image quality or resulting in the need to increase the dose of the radiation R. Thus, basically, the front cover 111 serving as the member forming the radiation incident surface 101 is often made of a resin material instead of a metal material. In this case, CFRP is desirable as the resin material for forming the front cover 111 from the viewpoints of robustness and weight. In the present exemplary embodiment, the thickness of the front cover 111 made of CFRP is 1.5 mm or thinner, more desirably, 1.0 mm or thinner.

The radiation incident surface 101 is a contact surface with the subject such as a patient and is also a surface through which the radiation R is transmitted, and thus basically has no large uneven shape and is formed by a flat surface. On the other hand, the recess-shaped grip portions 1121 for enabling the user to easily hold the radiographic imaging apparatus 100, a battery storage portion (not illustrated), and the like are provided on the rear surface 102 as illustrated in FIG. 1B.

Further, the user interface 120, including the power button and the connector for the power cable, is provided on the side surface 103 as illustrated in FIG. 1A. This means that an uneven shape, a step, a groove, and the like are formed on the rear surface 102 and the side surface 103.

The rear cover 112 forming the rear surface 102 and the frame 113 forming the side surface 103 have less influence on the transmittance of the radiation R, and thus may not necessarily be made of a resin material unlike the front cover 111 forming the radiation incident surface 101, except in cases of considering the weight. On the contrary, it is advantageous to make the rear cover 112 forming the rear surface 102 and the frame 113 forming the side surface 103 by using a metal material because this can prevent the emitted radiation R from entering the inside of the radiographic imaging apparatus 100 again by being reflected within an imaging room. In a case where a metal material is employed for the rear cover 112 forming the rear surface 102 and the frame 113 forming the side surface 103, the material is desirably as lightweight as possible and, for example, magnesium (Mg) or aluminum (Al) is suitable as the material. The present exemplary embodiment includes a configuration in which the rear cover 112 and the frame 113 serving as the members forming the rear surface 102 and the side surface 103 of the casing 110, respectively, are at least partially made of a metal material.

Next, the antibacterial agent to be applied when the antibacterial layer 301 is formed on at least a part of the surface of the casing 110 of the radiographic imaging apparatus 100 according to the present exemplary embodiment will be described.

The antibacterial agent in the present exemplary embodiment refers to a substance having at least an effect of suppressing multiplication of bacteria and viruses, and includes an agent exhibiting a sterilization effect. Various antibacterial agents, such as organic and inorganic types, are proposed as the antibacterial agent, but the inorganic type is desirable in consideration of chemical resistance and effect on human bodies. In this case, examples of the inorganic antibacterial agent include a titanium-based type, a silver-based type, a copper-based type, a zinc-based type, and a mercury-based type, but the titanium-based type, the silver-based type, and the copper-based type are particularly desirable in consideration of the viewpoints of the antibacterial effect and the use on the contact portion with the subject such as a patient.

Further, in recent years, a photocatalyst has been often used as the antibacterial agent. Especially, the development of titanium oxide-based antibacterial agents has been advanced, and an antibacterial agent exhibiting an antibacterial effect with not only ultraviolet light but also slight visible light has been developed (refer to Japanese Patent Application Laid-Open No. 2012-139690). Further, titanium oxide has less influence on human bodies. Moreover, even when used on the contact surface with the subject such as a patient, titanium oxide provides a less sticky texture and thus is suitable for use in the radiographic imaging apparatus 100. The types of titanium oxide include an anatase type, a rutile type, a brookite type, and an amorphous type, and the anatase type and the rutile type are desirable from the viewpoint of the antibacterial effect. In the present exemplary embodiment, in a case where titanium oxide is used as the antibacterial agent, the antibacterial agent includes not only titanium oxide itself but also a titanium oxide-based antibacterial agent. Further, in the present exemplary embodiment, titanium oxide supported by a porous member, such as hydroxyapatite, activated carbon, zeolite, and silica gel, may also be used as titanium oxide. Further, titanium oxide coated with resin such as silicone, or titanium oxide doped with sulfur may be used.

The antibacterial agent comes in various states such as powder and sol, but the antibacterial agent in sol state is suitable for the coating purpose in the present exemplary embodiment because the antibacterial agent is assumed to be applied in a state of being dispersed in a liquid.

FIGS. 3A to 3E illustrate examples of a configuration at and near the front cover 111, which is indicated by a broken line box B in FIG. 2, in the radiographic imaging apparatus 100 according to the present exemplary embodiment. In FIGS. 3A to 3E, similar components to those illustrated in FIGS. 1A, 1B, and 2 are denoted by the same reference numerals, and the detailed description thereof will be omitted. Each of FIGS. 3A to 3E also illustrates an XYZ coordinate system corresponding to the XYZ coordinate system illustrated in FIG. 2.

As indicated by each of the configuration examples illustrated in FIGS. 3A to 3E, the antibacterial layer 301 is formed on the radiation incident surface 101 side of the front cover 111 in the present exemplary embodiment. Further, the front cover 111 is formed using CFRP in the present exemplary embodiment. More specifically, FIG. 3A illustrates the configuration example in which only the antibacterial layer 301 is formed on the radiation incident surface 101 side of the front cover 111. FIG. 3B illustrates the configuration example in which a base layer 302 is interposed between the front cover 111 and the antibacterial layer 301 illustrated in FIG. 3A. FIG. 3C illustrates the configuration example in which a print layer 303 is interposed between the front cover 111 and the antibacterial layer 301 illustrated in FIG. 3A. FIG. 3D illustrates the configuration example in which the base layer 302 illustrated in FIG. 3B is interposed between the print layer 303 and the antibacterial layer 301 illustrated in FIG. 3C. FIG. 3E illustrates the configuration example in which an illustrative sheet 304 is interposed between the front cover 111 and the antibacterial layer 301 illustrated in FIG. 3A.

The present exemplary embodiment is characterized in that the average thickness of the antibacterial layer 301 formed on the radiation incident surface 101 side of the front cover 111 is thinner than 0.5 μm. The antibacterial layer 301 illustrated in FIGS. 3A to 3E is extremely thin in order to reduce contact between particles of the antibacterial agent applied in forming the antibacterial layer 301, thereby actively bring the antibacterial agent and the base material into contact with each other. In many cases, general antibacterial agents for use in coating applications are thermally cured to enhance adhesion by blending a curing agent and a reactive group in a solution in which the antibacterial agent is dispersed, or are heated at a high temperature to establish strong adhesion between the antibacterial agent particles without a curing agent. Examples of a method for curing the antibacterial agent include a method using ultraviolet light, but titanium oxide is not suitable for fixation using ultraviolet curing because titanium oxide absorbs ultraviolet light.

Applying heat to the members forming the casing 110 of the radiographic imaging apparatus 100, especially to the front cover 111 made of CFRP may cause contraction or a deformation, and thus heating such a member is not desirable. If the front cover 111 of the casing 110 of the radiographic imaging apparatus 100 is warped on the radiation incident surface 101 side, there may be a case where the edge of the side surface of the front cover 111 is lifted. The lifted edge of the side surface of the front cover 111 may cause an injury to the subject, such as a patient, touching the edge, or cause a gap between the front cover 111 and the frame 113 and result in entry of a disinfectant into the radiographic imaging apparatus 100 and also a leak of light from the gap. Especially, in a case where paint is applied to the front cover 111, the deformation caused by heating the front cover 111 has a significant influence due to a difference in heat contraction rate, and the paint may crack in some cases. Further, the thermal deformation depends on the thickness of the front cover 111. If the thickness of the front cover 111 is 1.5 mm or thinner, a thermal deformation is likely to occur even at a temperature of approximately 60° C. If the thickness of the front cover 111 is 1.0 mm or thinner, a deformation may occur even at a temperature of approximately 50° C. or lower. On the other hand, increasing the thickness of the front cover 111 leads to a failure to accommodate the components within the casing 110 and an increase in the weight. In light of this, in the present exemplary embodiment, the thickness of the front cover 111 is 1.5 mm or thinner, more desirably, 1.0 mm or thinner.

Further, in a case where no heat is applied when the antibacterial layer 301 is formed on the radiation incident surface 101 side of the front cover 111, the antibacterial agent particles having weak adhesion therebetween and failing to strongly adhere with the aid of, for example, an intermolecular force with the base material, can easily peel off. The radiographic imaging apparatus 100 of a mobile type (a portable type) is used in direct contact with the subject who is a patient, and thus the coating film strength of the antibacterial agent is especially important for the mobile type.

In the present exemplary embodiment, the average thickness of the antibacterial layer 301 formed on the radiation incident surface 101 side of the front cover 111 illustrated in FIGS. 3A to 3E is desirably thicker than 0.05 μm and thinner than 0.5 μm. More desirably, the average thickness of the antibacterial layer 301 formed on the radiation incident surface 101 side of the front cover 111 is 0.1 μm to 0.3 μm (0.1 μm or thicker and 0.3 μm or thinner). For example, if the average thickness of the antibacterial layer 301 formed on the radiation incident surface 101 side of the front cover 111 is thicker than 0.5 μm, the antibacterial layer 301 may fail. In other words, this condition causes damage to the external appearance of the radiographic imaging apparatus 100 and also makes it difficult to perform the antibacterial treatment of the surface of the casing 110 with sufficient coating film strength. On the other hand, if the average thickness of the antibacterial layer 301 formed on the radiation incident surface 101 side of the front cover 111 is thinner than 0.05 μm, the antibacterial agent may peel off in the form of a film, or a sterilization effect of ultraviolet light may be unable to be obtained in a case where titanium oxide is used as the antibacterial agent.

In other words, this condition causes damage to the external appearance of the radiographic imaging apparatus 100 and also makes it difficult to perform the antibacterial treatment of the surface of the casing 110 with sufficient coating film strength. Examples of a method for measuring the average thickness of the antibacterial layer 301 include a method that observes the cross section of the antibacterial layer 301 with an electron scanning microscope and calculates an average value of thicknesses at a plurality of points.

Further, in the radiographic imaging apparatus 100 according to the present exemplary embodiment, the adhesion strength of the antibacterial layer 301 is assumed to satisfy, for example, classification 2 in the cross-cut test defined in JIS-K5600-5-6.

Further, the use of titanium oxide as the antibacterial agent to be applied when the antibacterial layer 301 is formed has such an advantage that the antibacterial effect lasts a long time because titanium oxide is used as a solid, unlike silver ions and the like, and titanium oxide itself is not consumed, unlike silver ions. The radiographic imaging apparatus 100 is used over a plurality of years, and thus it is desirable to use titanium oxide capable of producing the long-lasting antibacterial effect, as the antibacterial agent to be applied when the antibacterial layer 301 is formed. Further, in the present exemplary embodiment, when the antibacterial layer 301 is formed on the surface of the casing 110, the surface is coated by applying the antibacterial agent thereto, and thus the surface can be coated again at the time of breakage due to unexpected use. CFRP in which an antibacterial agent is kneaded at the pre-impregnated (prepreg) stage (refer to Japanese Patent Application Laid-Open No. 2021-51069) is not desirable because the need to replace the expensive CFRP itself arises when the antibacterial effect vanishes.

Further, in the present exemplary embodiment, the average particle diameter of the antibacterial agent to be applied when the antibacterial layer 301 is formed is desirably 10 nm to 100 nm (10 nm or larger and 100 nm or smaller, or 0.01 μm or larger and 0.1 μm or smaller). More specifically, in the present exemplary embodiment, the antibacterial agent to be applied when the antibacterial layer 301 is formed is small in average particle diameter and thus is large in surface area, thereby making it possible to enhance the antibacterial effect even if the antibacterial layer 301 is thin. Pulverized titanium oxide used as a white pigment is not desirable as the antibacterial agent according to the present exemplary embodiment because the average particle diameter thereof is large and visible light is easily scattered thereby.

Further, in the present exemplary embodiment, the antibacterial layer 301 contains a metal material such as titanium oxide, but has an extremely small influence on the transmittance of the radiation R because the average thickness of the antibacterial layer 301 illustrated in FIGS. 3A to 3E is thinner than 0.5 μm. Further, because the average particle diameter is smaller than the wavelength of visible light, the antibacterial layer 301 has less influence on the visibility of the print layer 303, which is located on a more inner side than the antibacterial layer 301 in the configuration examples illustrated in FIGS. 3C and 3D.

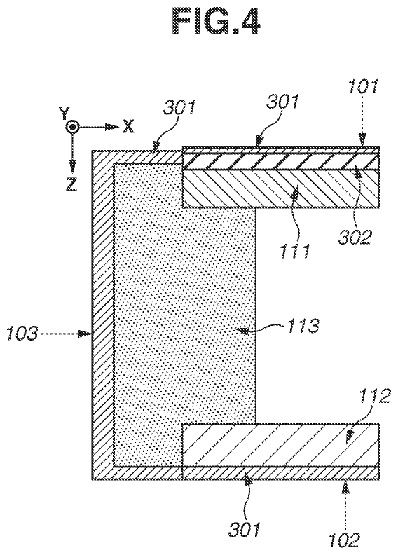

FIG. 4 illustrates an example of a configuration at and near the frame 113, which is indicated by a broken line box C in FIG. 2, in the radiographic imaging apparatus 100 according to the present exemplary embodiment. In FIG. 4, similar components to those illustrated in FIGS. 1A to 3E are denoted by the same reference numerals, and the detailed description thereof will be omitted. Further, FIG. 4 also illustrates an XYZ coordinate system corresponding to the XYZ coordinate system illustrated in FIG. 2.

As illustrated in FIG. 4, in the present exemplary embodiment, the antibacterial layer 301 is formed on the surface of the rear cover 112 forming the rear surface 102 and the surface of the frame 113 forming the side surface 103, in addition to the surface of the front cover 111 forming the radiation incident surface 101. In the example of FIG. 4, the average thickness of the antibacterial layer 301 on the surface of the rear cover 112 forming the rear surface 102 and the surface of the frame 113 forming the side surface 103 is thicker than the average thickness of the antibacterial layer 301 on the surface of the front cover 111 forming the radiation incident surface 101. More specifically, in the present exemplary embodiment, the average thickness of the antibacterial layer 301 on the surface of the front cover 111 forming the radiation incident surface 101 is desirably thicker than 0.05 μm and thinner than 0.5 μm as described above. In the present exemplary embodiment, the average thickness of the antibacterial layer 301 on the surface of the rear cover 112 forming the rear surface 102 and the surface of the frame 113 forming the side surface 103 is desirably thicker than 0.5 μm.

With the configuration according to the present exemplary embodiment, the antibacterial layer 301 with excellent abrasion resistance can be formed also on the frame 113 serving as the member forming the side surface 103 and the rear cover 112 serving as the member forming the rear surface 102, without applying heat thereto. As described above, the antibacterial layer 301 on the rear surface 102 and the side surface 103 has less influence on the transmittance of the radiation R even if being thicker than the antibacterial layer 301 on the radiation incident surface 101. If, for example, a printed marking requiring visibility is not present on the rear surface 102 or the side surface 103, the antibacterial layer 301 may not necessarily be thinly formed on the rear surface 102 or the side surface 103.

Further, the strength of the rear cover 112 serving as the member forming the rear surface 102 and the frame 113 serving as the member forming the side surface 103 is enhanced by provision of an uneven shape thereto, and this can reduce the deformation due to heating even when CFRP is used as the material. Further, if being made of a metal material, the rear cover 112 and the frame 113 can be heated. It is difficult to make the front cover 111 serving as the member forming the radiation incident surface 101, using a metal material as described above. On the other hand, it is possible to make the rear cover 112 and the frame 113 using a metal material. Thus, in a case where the rear cover 112 and the frame 113 are made of a metal material, the adhesion of the antibacterial agent can be improved by heating after the application of the antibacterial agent. Further, in this case, the rear cover 112 and the frame 113 can be corrected into an appropriate shape by annealing treatment.

The rear cover 112 and the frame 113 may not necessarily be entirely made of a metal material, and may be at least partially made of a metal material. For example, the rear cover 112 and the frame 113 may be partially formed of a metal material within a range capable of suppressing the deformation due to heating, and an area around the metal material may be thickened with a resin material by insert molding.

Further, the advantages of using the photocatalyst as the antibacterial agent according to the present exemplary embodiment include an effect of blocking ultraviolet light. Ultraviolet light (UV) is known to be capable of destroying bacteria and viruses but has an issue where a material irradiated with ultraviolet light is deteriorated. The photocatalyst is capable of absorbing ultraviolet light, and the antibacterial agent having extremely small particles is employed in the present exemplary embodiment. Thus, the penetration of ultraviolet light into the inside of the casing 110 can be hindered by reducing gaps between the particles to create a state in which the photocatalyst is densely distributed.

Referring now back to FIGS. 3A to 3E, the present exemplary embodiment will be further described.

As illustrated in FIG. 3C, the print layer 303 may be interposed between the front cover 111 and the antibacterial layer 301 in the present exemplary embodiment. By employing a white pigment containing rutile-type titanium oxide or a black pigment containing carbon black as paint for the print layer 303, the deterioration of the paint can be suppressed even if ultraviolet light passes through the antibacterial layer 301 and enters the inside of the casing 110.

Further, in the present exemplary embodiment, the casing 110 contains CFRP and the metal member robust against ultraviolet light in the base material, and thus has less deterioration and prevents ultraviolet light from reaching as far as the inside of the casing 110 in which the internal components including the substrate 160 are arranged.

The radiographic imaging apparatus 100 includes portions difficult to disinfect with alcohol, such as the electric contact with the battery and the connection portion with the power supply cable. Ultraviolet sterilization is effective for such portions. Disposing the antibacterial layer 301 according to the present exemplary embodiment near the electric contact enables the electric contact to be sterilized without alcohol disinfection.

The radiographic imaging apparatus 100 according to the present exemplary embodiment makes it possible to prevent multiplication of and infection with bacteria and viruses by using the antibacterial effect and the ultraviolet sterilization in combination.

In the present exemplary embodiment, the surface of the casing 110 is to be thinly coated with the antibacterial agent so that the antibacterial layer 301 illustrated in FIGS. 3A to 3E has an average thickness thinner than 0.5 μm as described above.

One method for achieving the extremely thin film thickness of the antibacterial layer 301 is a vaper deposition-based method conventionally used for semiconductors or the like, but this method leads to a significant cost increase and thus is not appropriate as the coating method for the radiographic imaging apparatus 100. Coating methods such as spin-coating, spraying, and dipping enable the application of the antibacterial agent at a relatively low cost, but it is difficult to achieve the film thickness of the antibacterial layer 301 thinner than 0.5 μm by simply applying the antibacterial agent.

In light of this, in a method for manufacturing the radiographic imaging apparatus 100 according to the present exemplary embodiment, a coating solution in which the antibacterial agent is dispersed in a solvent having a saturated vapor pressure of 1 mmHg or higher, more desirably, 10 mmHg or higher at a temperature of 20° C. is applied to the surface of the casing 110 when the antibacterial layer 301 is formed thereon. The saturated vapor pressure of 10 mmHg or higher enables the solvent to be evaporated quickly after the application at room temperature, and there is less concern that the antibacterial agent particles may aggregate when the solvent is evaporated or the solvent may remain without being evaporated. On the other hand, too fast evaporation causes non-uniform application, and thus the saturated vapor pressure at a temperature of 20° C. is desirably 10 mmHg.

Further, in the method for manufacturing the radiographic imaging apparatus 100 according to the present exemplary embodiment, a contact angle between the coating solution and a coating surface, which is a surface of the casing 110, is desirably 60 degrees or smaller when the antibacterial layer 301 is formed thereon. The contact angle within this range provides high wettability and enables the coating solution to quickly wet the surface and spread thereon at the time of the coating, thereby obtaining the thin uniform antibacterial layer 301. In contrast, if the contact angle is large, the amount of the coating solution is to be increased, thereby resulting in an increase in the film thickness of the antibacterial layer 301. Also in this case, the molecules of the solvent are likely to gather when the solvent is evaporated, thereby causing unevenness in the distribution of the antibacterial agent.

Further, in the method for manufacturing the radiographic imaging apparatus 100 according to the present exemplary embodiment, the coating solution desirably uses a solvent having a surface tension of 70 dyn/cm or lower, more desirably, 50 dyn/cm or lower at a temperature of 20° C. when the antibacterial layer 301 is formed. Reducing the surface tension of the coating solution in this manner can broaden choices of the material for forming the coating surface, thereby enabling the above-described contact angle to be adjusted to 60 degrees or smaller even with hydrophobic CFRP. Examples of the solvent to be used include ethanol, isopropyl alcohol, and ethyl acetate, but are not limited thereto. The solvent to be used may be one type of solvent, or two or more types of solvents mixed together.

Further, to apply the antibacterial agent to the surface of the casing 110, the antibacterial agent is to be dispersed as evenly as possible in the coating solution. In the present exemplary embodiment, water can be mixed in the solvent within a range that satisfies the above-described saturated vapor pressure and surface tension in order to increase the dispersibility of the antibacterial agent in the solution. Adding water in this manner enables the antibacterial agent to be applied to the base material while maintaining an even concentration distribution. The solution containing such an antibacterial agent is less viscous, and thus spin-coating or spray-coating is desirable from the viewpoint of the application of the low viscous solution, and spin-coating is more desirable from the viewpoint of the reduction in the film thickness.

Further, a dispersant may be added to the solution as another method for facilitating the dispersion of the antibacterial agent. In this case, examples of the dispersant include polyhydric alcohols such as polyether polyol and polyester polyol, fatty acid salt such as magnesium stearate, aliphatic amine, sulfonate, and polysiloxanes, but are not limited thereto.

Further, the temperature at which the surface of the casing 110 is coated with the antibacterial agent is desirably 60° C. or lower, more desirably, 50° C. or lower in order to suppress the deformation of the base material. Under a low-temperature environment, a temperature not causing aggregation or separation of the content is desirable and the antibacterial agent is desirably applied under an environment of, for example, 5° C. or higher, though this depends on the solvent and the blending agent.

Further, in the present exemplary embodiment, various materials other than the antibacterial agent can be blended together in the solution containing the antibacterial agent. In this case, examples of the blended materials include a stabilizer, a dispersant, a hydrophilizing agent, a viscosity modifier, and a pH modifier, but are not limited thereto. Further, a small amount of pigment may be blended in the solution so that the peel-off state of the antibacterial agent and the coating state can be checked.

Further, increasing the wettability between the coating solution and the coating surface, which is a surface of the casing 110, is important in order to thinly form the antibacterial layer 301 as described above. Thus, the present exemplary embodiment provides a configuration that improves the coatability by not only defining the properties of the coating solution to be used but also performing hydrophilic treatment of at least part of the surface of the casing 110 (the coating surface) to which the coating solution containing the antibacterial agent is to be applied. For example, the hydrophilicity can be improved by applying the hydrophilic treatment, such as plasma treatment or chemical treatment, to at least part of the surface of the casing 110 (the coating surface) before the coating solution containing the antibacterial agent is applied thereto. In a case where CFRP employed for a member forming the casing 110 is painted, the hydrophilicity can be increased by blending not only a surfactant agent and a hydrophilizing agent but also a hydrophilic compound in the paint. The range of the solvents usable from the viewpoint of the contact angle can be widened by increasing the hydrophilicity on the base material side in this manner.

Further, in the present exemplary embodiment, the base layer 302 may be additionally formed as illustrated in FIGS. 3B and 3D in order to reduce the surface tension of the surface (the coating surface) of the casing 110 to which the coating solution containing the antibacterial agent is to be applied. The use of the base layer 302 can improve the wettability of the surface to which the antibacterial agent is to be applied, without making an improvement to the base material or the paint, and thus is optimum as the hydrophilizing method.

In FIG. 3B, the base layer 302 is disposed between the front cover 111 made of CFRP and the antibacterial layer 301. In FIG. 3D, the base layer 302 is disposed between the print layer 303 and the antibacterial layer 301.

The base layer 302 is made of a hydrophilic material. The hydrophilic material employable for the base layer 302 is not particularly defined, but the material desirably includes polymer or metal having a hydrophilic group, an oxidized inorganic substance, or hydroxide as a structure thereof. Further, examples of the above-described polymer having a hydrophilic group include polymers having a silanol group, a carboxyl group, a hydroxy group, an oxyalkylene group, an amino group, a sulfone group, and the like, but are not limited thereto. Further, the thickness of the base layer 302 is not particularly defined, but the base layer 302 affects the absorption of the radiation R if being too thick, and thus the thickness thereof is desirably 10 μm or thinner. If the base layer 302 is heated when being applied, a deformation occurs in CFRP or the like forming the front cover 111, and thus it is important to apply the base layer 302 without heating, similarly to the antibacterial agent for use in forming the antibacterial layer 301.

Further, a layer that absorbs less visible light is desirable as the base layer 302 that is used on the radiation incident surface 101 side. In a case where the base layer 302 that can easily absorb or reflect visible light is used on the radiation incident surface 101 side, it is desirable to reduce the thickness of the base layer 302. Further, the base layer 302 may contain the above-described disperser and curing agent, a silane coupling agent, a surfactant agent, an ultraviolet absorbent, and/or the like.

The user interface 120 including the power switch and the connector for the power cable, and the battery (not illustrated) are disposed on the casing 110 of the radiographic imaging apparatus 100 as described above, and a groove or the like may be formed at the joint portion. It is difficult to form the antibacterial layer 301 thinner than 0.5 μm on this groove portion by applying the antibacterial agent using spin-coating, but the groove portion is less likely to come into contact with an object and thus the antibacterial layer 301 may not necessarily have the thickness described in the present exemplary embodiment in such a portion from the viewpoint of abrasion resistance.

Further, in FIG. 3E, the illustrative sheet 304 is disposed between the front cover 111 and the antibacterial layer 301. As illustrated in FIG. 3E, the illustrative sheet 304 may be used as the coating surface for applying the antibacterial agent that is used to form the antibacterial layer 301. In the example of FIG. 3E, at the time of sticking the illustrative sheet 304 to the front cover 111, the antibacterial layer 301 on the illustrative sheet 304 may crack or peel off. Thus, it is desirable to apply the antibacterial agent, which is used to form the antibacterial layer 301, to the illustrative sheet 304 in a state where the illustrative sheet 304 has already been stuck to the front cover 111.

As described above, the method for manufacturing the radiographic imaging apparatus 100 according to the present exemplary embodiment defines the film thickness (the average thickness) of the antibacterial layer 301, the wettability of the coating surface, and the vapor pressure of the coating solution. This makes it possible to form the antibacterial layer 301 with high coating film strength without heating at the time of application of the antibacterial agent that is used to form the antibacterial layer 301 on at least the radiation incident surface 101 of the radiographic imaging apparatus 100. Further, the radiographic imaging apparatus 100 can be provided without a defect such as a deformation on the surface of the casing 110 of the radiographic imaging apparatus 100.

As described above, in the radiographic imaging apparatus 100 according to the present exemplary embodiment, the antibacterial layer 301 is formed on at least a part of the surface of the casing 110, and the average thickness (σ) of the antibacterial layer 301 is thicker than 0.05 μm and thinner than 0.5 μm.

The above-described configuration makes it possible to provide the radiographic imaging apparatus 100 with the antibacterial treatment applied to the surface of the casing 110 with sufficient coating film strength, without impairing the external appearance of the radiographic imaging apparatus 100.

The above-described exemplary embodiment of the present disclosure is merely an example of how to embody the present disclosure when implementing the present disclosure, and the technical scope of the present disclosure shall not be construed limitedly by the exemplary embodiment. An exemplary embodiment of the present disclosure can be implemented in various manners without departing from the technical idea thereof or the main features thereof.

According to the exemplary embodiment of the present disclosure, it is possible to provide a radiographic imaging apparatus with antibacterial treatment applied to a surface of a casing thereof with sufficient coating film strength, without impairing the external appearance of the radiographic imaging apparatus.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of priority from Japanese Patent Application No. 2021-183885, filed Nov. 11, 2021, which is hereby incorporated by reference herein in its entirety.

What is claimed is:

1. A radiographic imaging apparatus comprising:
a radiation detector configured to convert incident radiation into an electric signal related to a radiographic image;
a casing containing the radiation detector; and
an antibacterial layer formed on at least part of a surface of the casing,
wherein an average thickness of the antibacterial layer is thicker than 0.05 μm and thinner than 0.5 μm.

2. The radiographic imaging apparatus according to claim 1, wherein the antibacterial layer is formed on at least a radiation incident surface of the casing on which the radiation is incident.

3. The radiographic imaging apparatus according to claim 2, wherein the antibacterial layer is further formed on a rear surface of the casing, the rear surface being located on a side opposite to a side on which the radiation incident surface is located, and a side surface of the casing with respect to the radiation incident surface, in addition to the radiation incident surface.

4. The radiographic imaging apparatus according to claim 3, wherein an average thickness of the antibacterial layer formed on the rear surface and the side surface is thicker than an average thickness of the antibacterial layer formed on the radiation incident surface.

5. The radiographic imaging apparatus according to claim 4,
wherein the average thickness of the antibacterial layer formed on the radiation incident surface is thicker than 0.05 μm and thinner than 0.5 μm, and
wherein the average thickness of the antibacterial layer formed on the rear surface and the side surface is thicker than 0.5 μm.

6. The radiographic imaging apparatus according to claim 3, wherein at least part of a member of the casing that forms the rear surface or the side surface is made of a metal material.

7. The radiographic imaging apparatus according to claim 2, wherein a member of the casing that forms the radiation incident surface is made of carbon fiber reinforced plastic (CFRP) with a thickness of 1.5 mm or less.

8. The radiographic imaging apparatus according to claim 2,
wherein a print layer is formed between a member of the casing that forms the radiation incident surface and the antibacterial layer, and
wherein paint used for the print layer contains titanium oxide or carbon.

9. The radiographic imaging apparatus according to claim 1, wherein an antibacterial agent included in the antibacterial layer contains titanium oxide.

10. The radiographic imaging apparatus according to claim 1, wherein an average particle diameter of an antibacterial agent included in the antibacterial layer is 0.1 μm or smaller.

11. The radiographic imaging apparatus according to claim 1,
wherein the antibacterial layer is formed by applying an antibacterial agent to the at least part of the surface of the casing, and
wherein the at least part of the surface of the casing to which the antibacterial agent is to be applied is subjected to hydrophilic treatment.

12. The radiographic imaging apparatus according to claim 1, wherein adhesion strength of the antibacterial layer satisfies classification 2 in a cross-cut test defined by Japanese Industrial Standards (JIS)-K5600-5-6.

13. A method for manufacturing a radiographic imaging apparatus, the radiographic imaging apparatus including a radiation detector configured to convert incident radiation into an electric signal related to a radiographic image and a casing containing the radiation detector, the method comprising:
forming an antibacterial layer on at least part of a surface of the casing; and
adjusting an average thickness of the antibacterial layer to be thicker than 0.05 μm and thinner than 0.5 μm.

14. The method according to claim 13, wherein the antibacterial layer is formed on at least a radiation incident surface of the casing on which the radiation is incident.

15. The method according to claim 14, wherein a member of the casing that forms the radiation incident surface is made of CFRP with a thickness of 1.5 mm or thinner.

16. The method according to claim 13, wherein the antibacterial layer is formed by applying, to the at least part of the surface of the casing, a coating solution in which an antibacterial agent is dispersed in a solvent having a saturated vapor pressure of 1 mmHg or higher at a temperature of 20° C.

17. The method according to claim 16, wherein the coating solution is a coating solution in which the antibacterial agent is dispersed in a solvent having a saturated vapor pressure of 10 mmHg or higher at a temperature of 20° C.

18. The method according to claim 16, wherein a contact angle between the coating solution and the at least part of the surface of the casing is 60 degrees or smaller.

19. The method according to claim 16, wherein a solvent having a surface tension of 70 dyn/cm or lower at a temperature of 20° C. is used for the coating solution.

20. The method according to claim 19, wherein a solvent having a surface tension of 50 dyn/cm or lower at a temperature of 20° C. is used for the coating solution.
